(19) United States
(12) Patent Application Publication
(10) Pub. No.: US 2010/0069759 A1
(43) Pub. Date: Mar. 18, 2010

(54) Method for the quantitative display of blood flow

(76) Inventors: Thomas Schuhrke, Munich (DE); Guenter Meckes, Munich (DE); Joachim Steffen, Westhausen (DE); Hans-Joachim Miesner, Aalen (DE); Frank Rudolph, Aalen (DE); Werner Nahm, Buehlerzell (DE)

Correspondence Address: ECKERT SEAMANS CHERIN & MELLOTT, LLC, U.S. STEEL TOWER, 600 GRANT STREET, PITTSBURGH, PA, 15219-2788, US

(21) Appl. No.: 12/462,036

(22) Filed: Jul. 28, 2009

(30) Foreign Application Priority Data
Jul. 28, 2008 (DE) 10 2008 040 804.2

(51) Int. Cl. A61B 6/00; G06K 9/00

(52) U.S. Cl. 600/476; 382/128

(57) ABSTRACT

A method for the quantitative representation of the blood flow in a tissue or vascular region is based on the signal of a contrast agent injected into the blood. Several individual images of the signal emitted by the tissue or vascular region are recorded and stored at successive points in time. For image areas of the individual images, the respective intensities of different points in time are compared and the maximum intensities of the signals are determined for these image areas. The maximum intensities are represented for these image areas.

### BACKGROUND OF THE INVENTION

The invention relates to a method for the quantitative representation of the blood flow in a patient.

Several methods for observing and determining the blood flow in tissue and vascular regions are known in which a chromophore such as indocyanine green, for example, is applied. The fluorescent dye can be observed as it spreads in the tissue or along the blood vessels using a video camera. Depending on the area of application, the observation can be non-invasive or in the course of surgery, for example via the camera of a surgical microscope.

Many methods are known, where only the relative distribution of the fluorescent dye in the tissue or in the blood vessels is examined qualitatively in order to draw conclusions concerning their blood flow. For example, conclusions are made about the blood flow and diagnoses are provided by watching an IR video recorded during surgery. It is also known to record an increase in the brightness of the fluorescence signal over time at all or at selected image points and in this manner record a time chart of the signal emitted by the fluorescent dye. The profile of the recorded formation plot provides the physician with information about potential vascular constrictions or other problems in this area of this image point. One example for this is provided in DE 101 20 980 A1. However, the method described in the DE 101 20 980 A1 goes beyond the qualitative analysis and embarks on a path towards a quantitative determination of the blood flow at every image point.

The objective forming the basis of the invention is to provide medical professionals with additional aids from which they can draw conclusions concerning blood flow problems and that can support making a diagnosis.

This objective, as well as other objectives which will become apparent from the discussion that follows, are achieved, according to the present invention, by the method and apparatus described below.

According to the invention, the contrast agent flowing into the tissue or vascular area is observed by recording the signal emitted by said contrast agent as a video, by splitting the video into individual images and storing the same, or by storing individual images directly, and by determining for several corresponding image areas, in particular image points of the individual images, the respective maximum achieved signal intensity, in order to generate a two-dimensional representation of the total flow, i.e., the maximum signal value achieved in all areas over the entire recorded time a vessel representation based on the maximum intensity determined for the image points. Because this maximum is reached when the maximum concentration of the contrast agent flowing through the blood vessels has arrived at an image point, this is achieved at different times at the various image points. For this reason, only the representation of the maxima reached at different points in time and thus visible in different individual images at different times on a combined representation provides an overview of the blood flow of all regions that is not possible when viewing the individual images. Until now, the physician had to view the recorded video several times in order to view the blood flow in different areas of the tissue or vascular region. This made it difficult to recognize if tissue areas had a poor blood flow or none at all. Due to the blood vessel representation according to the invention, the observer is able to recognize the maximum achieved concentration of the contrast agent at the same time at every point of the tissue or vascular region by viewing one single representation. In an ideal case, the respective image areas of the individual images can be the same local image point or image area, that is, for example, if the resolutions is reduced and image points are to be combined, a number of adjacent image points, if different individual images have been recorded with the same resolution of exactly the same detail of the object, or according to the invention in one advantageous embodiment can also be image points or image areas in different individual images that still are to be assigned to each other, because the recording conditions have changed between the recordings, for example, object and shooting direction have moved in relation to each other or the resolution has been changed or the like. This will be explained in greater detail in a later section. Preferably, the injected contrast agent is a fluorescent dye, such as indocyanine green, for example. However, other dyes known for perfusion diagnostics can be used as well. The excitation of the fluorescence for generating the signal to be obtained occurs typically via a near infrared light source. An infrared camera, which is often a CCD camera or a CMOS camera and which can be an autonomous medical device or can be integrated in a surgical microscope, is used for recording. The generation of the individual images of the signals that are to be recorded occurs either by splitting a video into individual images or directly through storing recorded individual images in certain time sequences. The individual images may be stored as a bitmap, for example. The maximum intensity for each image area or image point can be determined by comparing the intensity of across all individual images at each of the image areas or image points that are of interest. However, it is also possible to generate a plot as a function of the time for each image area or image point with the maximum of the plot constituting the maximum intensity.

In one advantageous embodiment, the minimum intensity per image area is determined and the maximum intensity is defined as the difference of Iₘₐₓ−Iₘᵢₙ. In this manner, the maximum intensity is purged of a potentially present residual fluorescence of previous examinations and only the concentration changes of the contrast agent are represented. The minimum intensity can again be defined via the intensity of a useful, selected individual image such as, for example, of the first individual image.

In an additional preferred embodiment a grayscale is selected as the scale for the blood vessel representation. This provides a clear. Quick overview about the blood flow in the tissue or in the vascular region and is easy to generate.

In one additional preferred embodiment, prior to determining the maximum intensity, a movement compensation is applied to the individual images. This means, the individual images are, if they are offset from each other, first placed on top of each such that indeed the respective associated image points that correspond to the same locations on the recording object are compared when determining the intensity. The underlying problem here is that the recording unit or the object to be recorded may move during recording. In such a case, the recorded images of the signals will be, at least slightly, shifted in relation to each other, such that this shift must first be reversed if one plans to receive a steady signal progression for each image point of the recorded object. Such a steady progression us a prerequisite for determining the maximum intensity for each image point. Thus, without movement compensation, the maxima could be assigned falsely to the image points and could lead to an erroneous representation of the total blood flow. Preferably, the movement is compensated using edge detection, where edge images of the individual images are generated that can then be correlated in order to determine from it the shift vector. As soon as the shift vector of an individual image is determined, this individual image is shifted in relation to the previous image according to the shift vector. In one embodiment, the edge images of successive individual images are used for the correlation of the edge images.

Preferably, however, the edge image of an individual image is correlated to a reference image that is generated by joining together the previous edge images of the individual images that have already been correlated to each other. In the course of this process, this creates a reference image that includes all the edges that have occurred in the individual images that have been correlated before. Any individual image can be used as the starting reference image, or an image where the total signal strength has exceeded a certain value or where it is determined in another fashion that the recorded signal has exceeded a noise level and is indeed the signal of the inflowing contrast agent. Generating the summed up reference image is essential because individual images that are recorded at very different times can shown a totally different edge structure because the signal may have already flattened in one area when it reaches the maximum in another area. It would then not be possible to properly correlate these very different images that have been recorded at different points in time.

In another advantageous embodiment, a brightness correction is applied to the individual images that takes into account changes in the recording conditions that affect the brightness of the signal. For example, the amplification factor at the camera can be adjusted such that a greater contrast range of the signal can be captured during recording. The intensity of the light source or other recording conditions can be adjusted as well such that the brightness correction may need to take several different parameters into account. For this purpose, changes in the recording conditions are stored together with the individual images, and during the brightness correction, the recorded signal values are converted to a common value range taking into account these stored data. This ensures that in the course of time a steady signal progression occurs at every image point.

For a full understanding of the present invention, reference should now be made to the following detailed description of the preferred embodiments of the invention as illustrated in the accompanying drawings.

### BRIEF DESCRIPTION OF THE DRAWINGS

FIG. 1 shows a schematic sequence of a method for presenting the blood flow.

FIG. 2 shows an example of a profile of a brightness plot at one image point.

FIGS. 3a and b show examples of blood vessel representations without and with movement compensation.

FIG. 4 shows schematically a surgical microscope for carrying out the method according to the invention.

### DESCRIPTION OF THE PREFERRED EMBODIMENTS

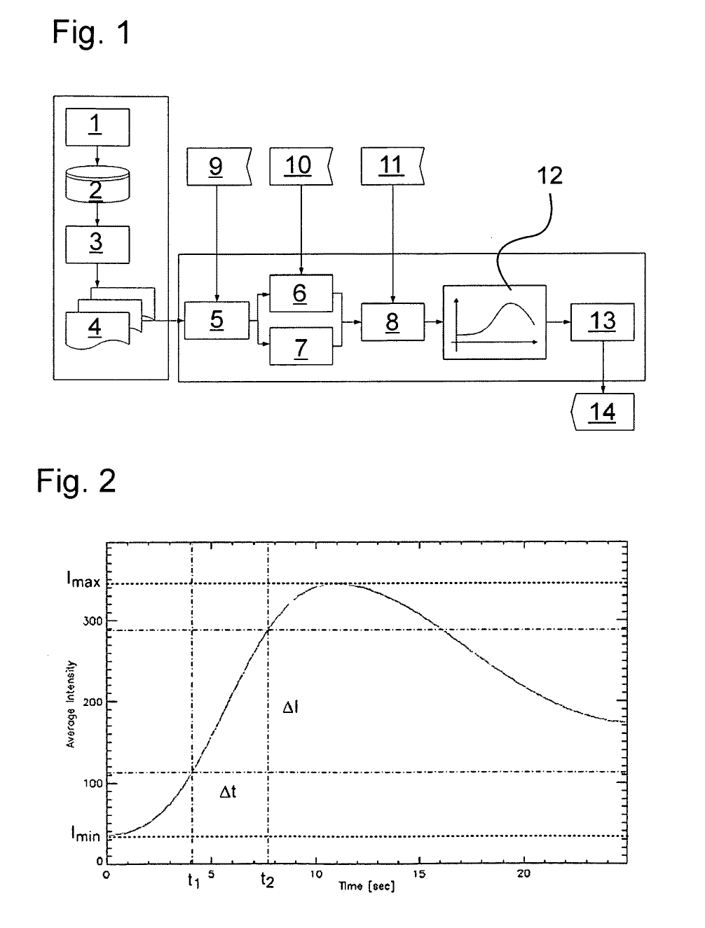

The preferred embodiments of the present invention will now be described with reference to FIGS. 1-4 of the drawings. Identical elements in the various figures are designated with the same reference numerals.

The complete system with the data flows and the individual processing steps is described in FIG. 1 and is used for presenting and evaluating the blood flow. The data are recorded using a video camera 1 in the infrared range, which is arranged at the surgical microscope—not shown—or is a component thereof. The recorded infrared videos are stored in a data memory 2 and are split into individual images 4 using a video player 3. Alternatively, it is also possible to store the images of the video camera 1 directly as individual images 4. A frequency of five frames 4 per second proved to be useful for this. They are then corrected in a single image correction step 5. In the process, the corrections for the edge drop, for the dark offset or of non-linearities of the video camera 1 are carried out taking into account the required correction data 9. The data of the corrected individual images 4 are than stored in the form of compressed binary data (e.g., Motion JPEG2000 Data (MJ2)) or in the form of non-compressed binary data (e.g., bitmap). In the form of non-compressed binary data, access times are shorter and the evaluation is faster.

For the evaluation, the individual images 4 are transferred to the algorithms for the brightness correction 6 and movement compensation 7. For the brightness correction 6, for example, the different amplification factors that have been set at the video camera 1 are taken into account during the recording of the video in order to adapt the video camera 1 to the different fluorescence strength of the tissue or vascular area to be recorded. They are documented during the recording as well, are stored on the data memory 2 as metadata 10 assigned to the video data and are computed with the individual images 4. During the movement correction 7, the positions of the recorded individual images 4 are aligned. The video camera 1 or the object, i.e., the tissue or vascular area to be recorded may move during video recording. In such cases, the individual images 4 are offset from each other. Thus, the individual images 4 must be re-aligned in order to evaluate the details visible in the individual images 4 without faults. This is exacerbated by the constantly changing image information in the individual images 4. To have an initial image for comparison purposes, a reference image is selected from among the individual images 4. The first image on which clear structures can be recognized can serve as an initial reference image. Using an edge detection method, all additional individual images 4 that are to be computed with the reference image are continuously examined for their degree of offset in comparison to the reference image. This offset is taken into account in all additional steps where several individual images 4 are involved. In particular the reference image is continuously updated by integrating the edge image of the following individual image that is offset to the correct position into the reference image.

The brightness determination 8 can be carried out following the corrections 6 and 7. For this purpose, first the position of the measurement range is determined in a measurement range determination 11. The measurement range for which the blood vessel representation is to be generated can be defined in a measurement range determination 11 via a measurement window or can be a selection of specified measurement points. For example, a range of the recording can be selected if a blood vessel representation is desired for this range only, or the blood vessel representation is generated for a portion of the image points only in order to save computing time. The result of the brightness determination 8 is a brightness plot 12 as a function of the time as can be seen in FIG. 2. This brightness plot 12 is computed for all or at least for a sufficiently large sample of image points.

In an evaluation 13, numerous other representations 14, comprising individual results as well, can be supplied from these brightness plots 12 and the individual images 4. They can then be represented on the screen together with the individual images 4.

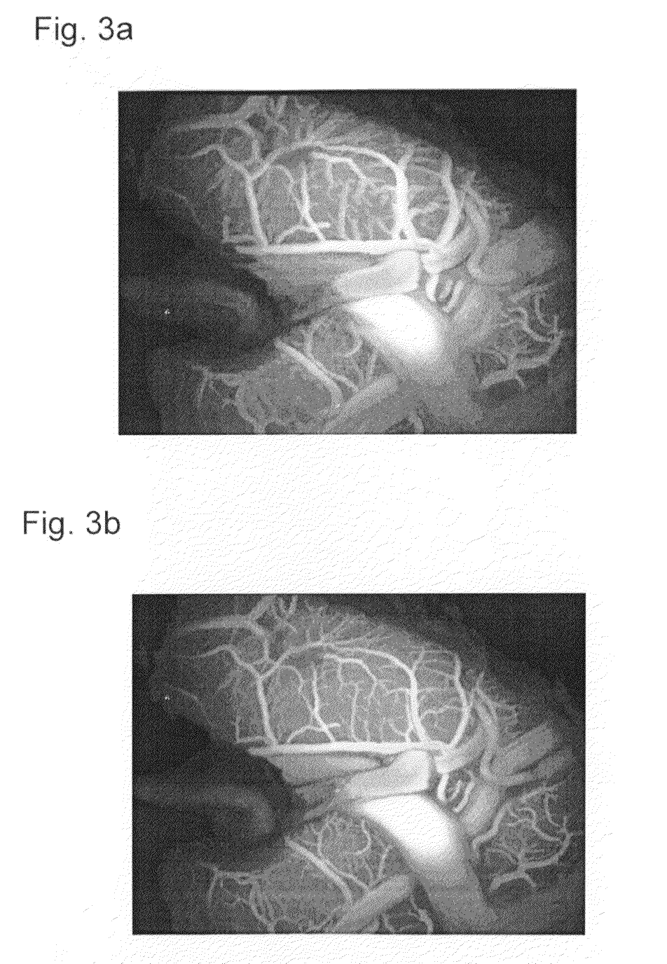

On example for this is a so-called blood vessel representation, where all vessels in which fluorescent agents have flowed and all tissues through which fluorescence agents flowed appear light. This representation is generated by determining and representing the maximum and brightness value for each image point of the brightness and movement corrected individual images 4. With this maximum brightness for each image point, one obtains a relative, quantitative quantity for the blood flow at all positions. These maximum brightnesses are scaled and represented as a grayscale image. This type of representation enables the physician to recognize defects more easily. Examples for blood vessel representations can be seen in FIGS. 3a and 3b. FIG. 3a shows a blood vessel representation that has been generated without movement compensation 7, while FIG. 3b shows an example with movement compensation 7. Clearly recognizable is the significantly better sharpness of the contours in FIG. 3b with movement compensation.

As an alternative to the maximum brightness, it is also possible to represent the contrast Iₘₐₓ−Iₘᵢₙ, the difference between the maximum and minimum brightness value. This shows the maximum change in the contrast agent concentration.

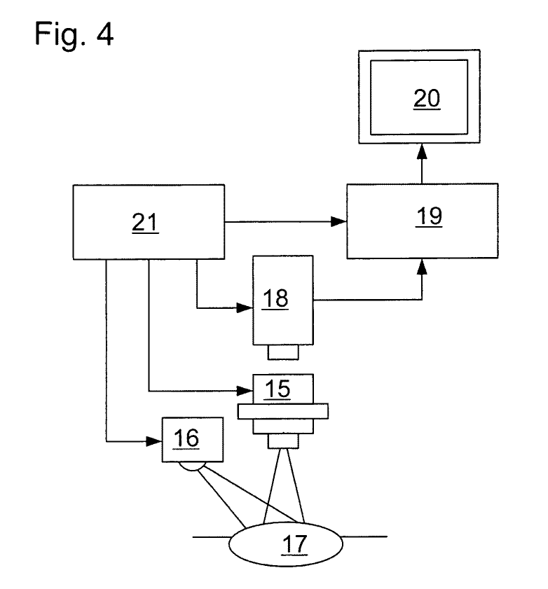

FIG. 4 shows schematically the essential components of a surgical microscope that can be used to apply the method according to the invention. The optics 15 of a surgical microscope reproduces an object 17, for example the head of a patient that is to be treated during surgery and is illuminated by a light source 16 of the surgical microscope in a camera 18. The camera 18 can also be a component of the surgical microscope. The image data recorded by the camera 18 are transferred to a computer unit 19 where they are evaluated. Medical quantities derived at the evaluation are then represented on the screen 20, potentially together with the recorded image. Similar to the computer unit 19, the screen 20 can be a component of the central surgical control but can also be a component of the surgical microscope. A control unit 21 controls the brightness of the light source 16 as well as the magnification factor and the aperture of the optics 15 and the amplification factor of the camera 18. In addition, the control unit 21 generates metadata that provide information about changes in the recording conditions that occur as soon as the control unit 21 adjusts a quantity that is to be controlled. These metadata are transferred from the control unit 21 to the computer unit 19, where they are assigned to the image data that have been provided to the computer unit 19 by the camera 18. Metadata and image data are stored, at least temporarily, by the computer unit 19 and are evaluated according to the method according to the invention. During the evaluation, the metadata are included with the image data. The results of the evaluation according to the invention are then displayed on the display unit 20, possibly together with the image data.

There has thus been shown and described a novel method and apparatus for the quantitative display of blood flow which fulfills all the objects and advantages sought therefor. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

What is claimed is:

1. A method for the quantitative representation of the blood flow in a tissue or vascular region based on the signal of a contrast agent injected into the blood, said method comprising the steps of:
recording and storing, at successive points in time, several individual images of the signal emitted by the tissue or vascular region are,
for image areas of individual images comparing their respective signal intensities in individual images that have been recorded at different points in time,
determining the maximum intensity of the signal for each of these compared intensities of the signal, and
representing the maximum intensities of the signal together for these image areas.

2. A method for the quantitative representation of the blood flow in a tissue or vascular region as set forth in claim 1, wherein a plot of the signal as a function of the time is obtained for each of the image areas to be viewed in order to determine the maximum intensity of the signal.

3. A method for the quantitative representation of the blood flow in a tissue or vascular region as set forth in claim 1, wherein the minimum intensity of the signal is subtracted from the maximum intensity of the signal.

4. A method for the quantitative representation of the blood flow in a tissue or vascular region as set forth in claim 1, wherein the maximum intensities of the signal for the image areas are represented in the form of a grayscale image.

5. A method for the quantitative representation of the blood flow in a tissue or vascular region as set forth in claim 1, wherein a movement compensation is applied to the individual images prior to the determination of the maximum signal intensity.

6. A method for the quantitative representation of the blood flow in a tissue or vascular region as set forth in claim 5, wherein edge images of individual images are generated for the movement compensation using an edge detection method.

7. A method for the quantitative representation of the blood flow in a tissue or vascular region as set forth in claim 6, wherein edge images are correlated to each other in order to determine a shift vector.

8. A method for the quantitative representation of the blood flow in a tissue or vascular region as set forth in claim 7, wherein each correlation of the edge image of an individual image is carried out using a reference image that is developed by supplementing said edge image with the current shifted edge image.

9. A method for the quantitative representation of the blood flow in a tissue or vascular region as set forth in claim 1, wherein a brightness correction is applied to the individual images prior to the determination of the maximum signal intensity.

10. A method for the quantitative representation of the blood flow in a tissue or vascular region as set forth in claim 9, wherein metadata are recorded and stored for the brightness correction during recording of the individual images.

11. A surgical microscope for recording a fluorescence radiation of a contrast agent comprising a camera for recording an image sequence of an object and optics for reproducing the object in the camera, wherein the camera is connected to a computer unit for deriving medical quantities from an image sequence of medical image data or individual images of the image sequence, the improvement wherein the computer unit operates in accordance with a program for carrying out the method according to claim 1.

12. An analysis system of a surgical microscope for recording a fluorescence radiation of a contrast agent, comprises a computer unit that operates in accordance with a program for performing the method as set forth in claim 1.
